(19) United States
(12) Patent Application Publication
(10) Pub. No.: US 2004/0235964 A1
(43) Pub. Date: Nov. 25, 2004

(54) Clear propofol compositions

(76) Inventors: Srikanth Annappa Pai, Maharashtra (IN); Sangeeta Hanurmesh Rivankar, Maharashtra (IN); Shilpa Sudhakar Kocharekar, Maharashtra (IN)

Correspondence Address: Garron M Hobson, Thorpe North & Western, PO Box 1219, Sandy, UT, 84091-1219, US

(21) Appl. No.: 10/250,420

(22) PCT Filed: Dec. 5, 2001
(86) PCT No.: PCT/IN01/00213

(30) Foreign Application Priority Data
Dec. 7, 2000 (IN) 1106/MUM/2000

(51) Int. Cl. A61K 31/05

(52) U.S. Cl. 514/731

(57) ABSTRACT

The present invention relates to a stable clear sterile aqueous composition of Propofol suitable for parenteral administration and a process for making the same. The composition comprises Propofol, TPGS and water. The composition of present invention gives clear product suitable for parenteral administration overcoming the disadvantages of emulsion formulation. The ratio of propofol to TPGS is at least 1:10 (by wt.) and the content of TPGS is from 1 to 20% w/v in the composition. The composition is rendered sterile by end autoclaving.

### FIELD OF INVENTION

This invention relates to a process for preparation of a clear, sterile anaesthetic composition of Propofol suitable for parenteral administration.

### BACKGROUND OF THE INVENTION

Propofol (2, 6-diisopropyl phenol) is an intravenous anesthetic agent characterised by a short recovery time. It has the desirable property of rapid onset and offset of the anaesthetic effect following intravenous administration and minimal accumulation on long-term administration.

Propofol even though is a preferred anesthetic agent, has posed a big challenge to the formulators since its invention because of its aqueous insolubility. It was at first formulated as a 1% aqueous solution containing nonionic surfactant Cremophor EL as a solubiliser. However Cremophor EL has been implicated in some adverse reactions when administered intravenously, including anaphylactoid reactions. Subsequently, the anesthetic agent was formulated as an oil-in-water emulsion containing 1% w/v Propofol with 10% w/v soybean oil and 1.2% w/v purified egg phosphatide.

Lipid based emulsions, however, suffer from several limitations which are as follows.

Improper storage results in poor physical stability.

On storage produces free fatty acid which is toxic.

It has potential to cause embolism because of higher oil globule size.

Causes pain on injection (contains oil in the formulation).

During administration online microbial filter cannot be used.

Cannot be visually inspected for foreign particles before administration as the product has milky appearance.

Can be selectively admixed with only a few injectable products before administration.

During administration, strict aseptic techniques must be followed as emulsion system is highly susceptible for bacterial growth.

During maintenance anaesthesia, infusion tubes require to be changed frequently as the product inside infusion tube may support bacterial growth.

Contains Egg lecithin as an emulsifier. Being from an animal source, may produce allergy in some patients.

Increases concentration of plasma phospholipid, decreases clearance of triglycerides causing hypertriglyceridemia, hypercholesterolemia.

Before autoclaving, the product cannot be filtered through 0.22μ filter.

The inherent limitations of the emulsion formulation as mentioned above could be overcome by eliminating vegetable oil and phospholipids from the formulation. Development of a composition of this type of Propofol formulation (removing vegetable oil and phospholipids) for parenteral administration is possible if Propofol is made soluble in aqueous phase using a solubiliser, as Propofol is an oily liquid, insoluble in water.

WO00/78301 describes an anaesthetic composition for intravenous injection containing Propofol and Poloxamer as the surfactant. Optionally it can contain at least one more surfactant selected from the group consisting of Solutol HS15, Egg lecithin, Labrasol, Polyoxy-10-oleyl ether, Tween, Ethanol and Polyethylene glycol. The composition is prepared in the form of a microemulsion having particle size below 100 nm and can be aseptically filtered.

WO01/64187 also describes an similar anaesthetic composition for intravenous injection containing Propofol and Poloxamer. This invention defines the type of polymers that can be used i.e. Propylene oxide portion is at least 2000D while the Ethylene oxide portion is at least 40% w/w. Under formulation stability, the influence of pH and added electrolyte (sodium chloride) on the hydrodynamic radii of micelles has been discussed. However, the stability of the active ingredient Propofol has not been considered in above two patents Poloxamer has been reported to be incompatible with phenols. Chef Martindale 32^{nd} edition, Pg. 1326) and hence it is doubtful that such compositions would retain propofol activity for a long time.

The main object of the present invention is to develop a clear, stable sterile aqueous composition of Propofol overcoming drawbacks of the existing formulation and also the drawbacks of the prior art.

### SUMMARY OF THE INVENTION

Accordingly the present invention relates to a clear stable anaesthetic composition suitable for parenteral administration comprising Propofol (1 mg/ml to 20 mg/ml of the composition), d-Alpha Tocopheryl Polyethylene Glycol 1000 Succinate (TPGS) keeping the ratio of propofol to TPGS at least 1:10 (by wt.) and the content of TPGS from 1 to 20% w/v, in the composition, and water with or without parenterally acceptable conventional additives.

The process of manufacturing the anaesthetic composition of present invention comprises

a) dissolving TPGS in water to give TPGS solution;

b) adding propofol under mixing to said TPGS solution to give anaesthetic composition;

c) said additives if required may be added to water, TPGS solution or anaesthetic composition formed;

d) making up the volume with water to the desired level of propofol in the anaesthetic composition;

e) filtering the composition obtained at the end of step (d) through 21μ and 0.2μ filter;

f) filling the filtrate obtained at the end of step (e) in containers such as vials, ampoules, plastic containers followed by nitrogen purging and sealing the filled containers;

g) autoclaving the sealed containers filled with said filtrate.

### DETAILED DESCRIPTION OF EMBODIMENTS OF THE INVENTION

While developing an anaesthetic composition of Propofol in light of the object set as above, we found that some hydrophobic therapeutic drugs are dissolved in α-tocopherol, an oily material and then the α-Tocopherol solution is emulsified using TPGS as emulsifier (Ref. W098/30205). As we intended to avoid oily composition, after lot of experimentation we found that by using sufficiently large quantities of TPGS we can solubilise the anaesthetic hydrophobic propofol in water giving a composition suitable for parenteral administration.

WO98/30205 discloses substantially ethanol-free self-emulsifying drug delivery system. The pharmaceutical compositions of the invention are typically formed by dissolving a therapeutic agent in ethanol to form a therapeutic agent solution. Alpha-tocopherol is then added to the therapeutic agent solution to form an alpha-tocopherol and therapeutic agent solution. Next, the ethanol is removed to a form a substantially ethanol-free alpha-tocopherol and therapeutic agent solution. The substantially ethanol free alpha tocopherol and therapeutic agent solution is blended with and without an aqueous phase incorporating a surfactant to form a pre-emulsion. For intravenous delivery the pre-emulsion is then homogenised to form a fine emulsion.

In this delivery system ethanol is used to dissolve the therapeutic agent and incorporated into large amount of α-tocopherol which is essential for holding the drug in solution.

TPGS is a water soluble form of alpha-tocopherol prepared by esterifying the acid group of crystalline d-alpha-tocopheryl acid succinate with polyethylene glycol 1000. TPGS is very stable and does not hydrolyze under normal conditions.

TPGS melts at 41° C. and degrades at temperatures above 199° C. TPGS has a solubility of 20 gm % in water at 20° C. TPGS has an HLB value in the region of 15- 19.

For dissolving TPGS in water, TPGS is required to be added to hot water. The preferred temperature of water could be from 45° C. to 100° C.

TPGS does not degrade if exposed to oxygen, heat, light or oxidising agents. It is unstable to alkali.

While preparing the composition of our present invention, after lot of experimentation we found that an oil soluble anaesthetic agent Propofol can be solubilised in water with the help of TPGS, without using any tocopherol or any oily phase if we use higher amounts of TPGS to give a solubilised product containing Propofol

The composition of present invention gives a stable product when stored under controlled conditions of temperature i.e. under refrigeration.

The Propofol content of the composition of present invention is from about 1 mg/ml to about 20 mg/ml, preferably from about 2 mg/ml to about 20 mg/ml, more preferably 10 mg/ml.

The TPGS content of the composition of present invention is from about 10 mg/ml to about 200 mg/ml, preferably from about 100 mg/ml to about 150 mg/ml, more preferably 100 mg/ml.

The present invention gives a clear stable, sterile aqueous composition suitable for parenteral administration without addition of any conventional additives.

However, in another embodiment of the invention addition of conventional additives if required by parenteral dosage form may be added to the aqueous solution at any stage of preparation.

Parenterally acceptable conventional additives when added are selected from a group of additives such as buffers, tonicity modifying agents, preservatives, antioxidants at conventional usage levels.

Any of the parenterally used buffer can be used in the composition of present invention which is selected from a group of buffers such as phosphate buffer, glycine buffer, citrate buffer or a mixture thereof. Phosphate buffer is preferred whenever buffer is used. Phosphate buffers with different compositions of sodium dihydrogen phosphate, disodium hydrogen phosphate, phosphoric acid, sodium hydroxide can be used to give a desired pH to the composition. A pH range of 4.0- 8.0 of the final composition is preferred.

The composition of present invention can also contain tonicity modifying agent to make the composition isotonic with the blood. The tonicity modifying agent is selected from a group of partenterally acceptable compounds such as dextrose, sodium chloride, mannitol, sorbitol, glycerin, propylene glycol or a mixture thereof Glycerin is particularly preferred as tonicity modifying agent at a concentration of 2- 3% w/v of the composition when used. 2.25% w/v of the glycerin is more preferred in the final composition

Preservatives when used in the composition of present invention are selected from a group of parenterally acceptable compounds such as disodium edetate, benzyl alcohol, sodium benzoate or a mixture thereof. Disodium edetate is the preferred preservative in a concentration range of 0.0025% to 0.01% w/v of the composition when used.

The composition of present invention can also contain antioxidants which are selected from a group of parenterally acceptable compounds such as ascorbyl-6 palmitate, ascorbic acid, salts of ascorbic acid. One of the advantages of TPGS is that it by itself acts as an antioxidising agent and additional antioxidants may not be necessary.

Various modifications, additions will become apparent to those skilled in the art in light of the foregoing disclosures without departing from the spirit and scope of the disclosed invention. Therefore it is to be understood that the invention is not limited to the disclosed embodiments, but may be practised within the full scope of claims appended herewith.

### Examples

The invention will now be illustrated by way of examples. The examples are by way of illustration only and in no way restrict the scope of the invention.

All the raw materials used in this example were of parenteral grade. Equipments used were of conventional nature. Entire processing was done in an area with a controlled environment. Nitrogen cover was provided while processing the batch.

The following materials were used in the Examples

a) Propofol was procured from Cilag AG

b) TPGS complying with United States National Formulary (USNF) specification was procured from M/s. Eastman Chemical Ltd.

c) Water complying with “Water for Injection” specifications of Indian Pharmacopoeia was used.

d) Glycerin complying with specifications of Indian Pharmacopoeia was used.

e) Disodium edetate complying with specifications of Indian Pharmacopoeia was used.

f) Potassium dihydrogen phosphate complying with specifications of USNF was used.

g) Disodium hydrogen phosphate complying with specifications of Indian Pharmacopoeia was used.

h) Diprivan—Propofol Emulsion (10 mg/ml) available commercially was used.

### Example I

TPGS was melted and 20 gms of molten TPGS was added to boiling water (170 ml). Stirred till TPGS got dissolved completely. Propofol (2 gms) was added to TPGS solution under stirring. The product was stirred till a clear solution was obtained. Volume was made upto 200 μl with water. Filtration was carried out using 2μ prefilter and 0.221μ membrane filter. The product was filled into sterile pyrogen free glass vials under nitrogen cover under laminar flow. The vials were closed using flurotec rubber stoppers and sealed using aluminium seals. The filled and sealed vials were autoclaved at 121° C. for 20 minutes.

STABILITY DATA OF PRODUCT OF EXAMPLE I UNDER
RECOMMENDED STORAGE CONDITIONS of 2-8° C.

| PERIOD | APPEARANCE | PROPOFOL CONTENT |
|---|---|---|
| Initial | Clear slightly yellowish liquid | 101.63% |
| 6 Months | Clear slightly yellowish liquid | 100.42% |
| 9 Months | Clear slightly yellowish liquid | 99.47% |

The above stability data indicates that the product prepared in Example I is suitable for commercial marketing.

### Example II

Example II was same as Example I except that Glycerin (4.5 gms) was added after solubilising Propofol.

### Example III

Example III was same as Example II except that Disodium Edetate (0.011 gms) was dissolved in boiling water before addition of TPGS.

### Example IV

Example IV was same as Example I except that Phosphate buffer of pH 5.5 was used in place of water.

Phosphate buffer of pH 5.5 was prepared by mixing 96.4 ml of “Solution I” with 3.6 ml of “Solution II”.

1) Solution I—Dissolve 13.61 gm of Potassium dihydrogen phosphate in sufficient water to produce 1000 ml.

2) Solution II—Dissolve 35.81 gm of Disodium hydrogen phosphate in sufficient water to produce 1000 ml.

### Example V

Same as Example I except that 16 gms of TPGS was used instead of 20 gms. The product obtained was hazy indicating incomplete solubilisation of propofol.

### Example VI

Propofol composition prepared in Example I was subjected to in-vivo toxicity studies in mice.

Single Dose Toxicity Study in Mice

Material and Method

Test System: Female Swiss albino mice in the weight range of 20-22 gm were obtained from the animal house of Bharat Serums & Vaccines Ltd (BSVL) and employed for the study. The animals were provided with standard chow and Aquaguard™ water, ad libitum.

Test Material: Propofol composition (10 mg/ml) prepared in Example I, was administered intravenously.

Comparative material: Diprivan—Propofol emulsion (10 mg/ml) available commercially was administered intravenously.

All animals (8 from each group) were observed for signs of clinical toxicity if any and for mortality for a period of 72 hours. The percent mortality was calculated for all the doses. The results are given in the following table.

| Dose | % mortality in sample of | % mortality in Diprivan |
|---|---|---|
| (mg/kg body wt.) | Example I | sample |
| 35 | 0 | 0 |
| 40 | 25 | 25 |
| 45 | 37.5 | 37.5 |
| 50 | 50 | 50 |

Observations

The groups which received Propofol composition from Example I showed similar signs of toxicity as compared to the group which received Diprivan.

### Example VII

Propofol composition prepared in Example I was subjected to efficacy study in mice.

Material and Method

Test System: Female Swiss albino mice in the weight range of 20-22 gm were obtained from the animal house of Bharat Serums & Vaccines Ltd (BSVL) and employed for the study. The animals were provided with standard chow and Aquaguard™ water, ad libitum.

Test Material: Propofol composition (10 mg/ml) prepared in Example I, was administered intravenously at a dose of 35 mg/kg.

Comparative material : Diprivan—Propofol emulsion (10 mg/ml) available commercially was administered intravenously at a dose of 35 mg/kg.

All the animals (8 from each group) were observed for the time taken to go into anaesthesia and the time taken to come out of anaesthesia. The observations are as follows:

| | |
|---|---|
| AVERAGE | AVERAGE |
| INDUCTION TIME | RECOVERY TIME |

| Example I | Diprivan | Example I | Diprivan |
|---|---|---|---|
| 4.2 sec. | 4.1 sec. | 8.6 min. | 7.9 min. |

The stability studies, toxicity studies and efficacy studies on Example I demonstrated that the composition of present invention is stable overcoming all the disadvantages of the emulsion formulation discussed earlier and comparable to Diprivan in toxicity and efficacy.

Advantages of the Present Invention:

The present invention gives a clear sterile anaesthetic composition that overcomes the disadvantages of emulsion formulation discussed earlier and gives a composition which has many advantages some of which are as follows:

i. The composition is clear, can be visually inspected before administration and can be administered with the use of on-line microbial filter.

ii. The composition does not contain phospholipids. Hence plasma phospholipids are unaffected on parenteral administration of the composition.

iii. The composition does not cause any change in triglyceride clearance

iv. The composition can be mixed with any of the commonly used diluents before administration.

1. A clear stable anesthetic composition suitable for parenteral administration comprising Propofol in an amount of about 1 mg/ml to about 20 mg/ml of the composition; d-Alpha Tocopheryl Polyethylene Glycol 1000 Succinate (TPGS) in an amount of from about 1 to about 20% w/v of the composition; water; and optionally one or more parenterally acceptable additives, wherein the ratio of propofol to TPGS at least 1:10 (by, wt.).

2. A clear stable anesthetic composition suitable for parenteral administration as claimed in claim 1 wherein the content of the Propofol is about 10 mg/ml of the composition.

3. A clear stable anesthetic composition suitable for parenteral administration as claimed in claim 1 wherein the content of TPGS is from about 100 mg/ml to about 150 mg/ml of the composition.

4. A clear stable anesthetic composition suitable for parenteral administration as claimed in claim 1 wherein the parenterally acceptable additives include at least one member selected from the group consisting of buffers, tonicity modifying agents, preservatives, antioxidants.

5. A clear stable anesthetic composition suitable for parenteral administration as claimed in claim 4, wherein the buffer is a parenterally acceptable buffer selected from the group consisting of phosphate buffers, glycine buffers, citrate buffers and mixtures thereof.

6. A clear stable anesthetic composition suitable for parenteral administration as claimed in claim 4, wherein the tonicity modifying agent is a parenterally acceptable compound selected from the group consisting of dextrose, sodium chloride, mannitol, sorbitol, glycol and mixtures thereof.

7. A clear stable anesthetic composition suitable for parenteral administration as claimed in claim 4, wherein the tonicity modifying agent is glycerin.

8. A clear stable anesthetic composition suitable for parenteral administration as claimed in claim 4, wherein the tonicity modifying agent is propylene glycol.

9. A clear stable anesthetic composition suitable for parenteral administration as claimed claim 4, wherein the preservative is a parenterally acceptable compound selected from the group consisting of disodium edetate, benzyl alcohol, sodium benzoate and mixtures thereof.

10. A process for preparation of clear stable anesthetic composition suitable for parenteral administration as claimed in claim 1, comprising
a) dissolving TPGS in water to give TPGS solution;
b) adding propofol under mixing to said TPGS solution to give an anesthetic composition;
c) optionally adding said additives;
d) adjusting the composition volume with water to attain a desired propofol concentration;
e) filtering the composition obtained at the end of step (d) through 2 μ and 0.2 μ filter;
f) filing the filtrate obtained at the end of step (e) into a container followed by nitrogen purging and sealing the filled container;
g) autoclaving the sealed container filled with said filtrate.

11. A clear stable anesthetic composition suitable for parenteral administration as claimed in claim 1 prepared by a process comprising
a) dissolving TPGS in water to give TPGS solution;
b) adding propofol under mixing to said TPGS solution to give an anesthetic composition;
c) optionally adding said additives;
d) adjusting the composition volume with water to attain a desired propofol concentration;
e) filtering the composition obtained at the end of step (d) through 2 μ and 0.2 μ filter;
f) filing the filtrate obtained at the end of step (e) into a container followed by nitrogen purging and sealing the filled container;
g) autoclaving the sealed container filled with said filtrate.
